(19) United States
(12) Patent Application Publication
(10) Pub. No.: US 2014/0105958 A1
(43) Pub. Date: Apr. 17, 2014

(54) EDIBLE STICKER WITH NUTRIENT

(71) Applicant: Bob D. NTOYA, North Hills, CA (US)

(72) Inventor: Bob D. NTOYA, North Hills, CA (US)

(21) Appl. No.: 14/054,818

(22) Filed: Oct. 15, 2013

Related U.S. Application Data
(60) Provisional application No. 61/712,979, filed on Oct. 12, 2012.

(51) Int. Cl. A23L 1/30; A61K 9/00; A23L 1/304; A23L 1/303; A23L 1/302

(52) U.S. Cl. CPC A23L 1/30; A23L 1/303; A23L 1/3014; A23L 1/302; A23L 1/304; A23L 1/3008; A61K 9/00
USPC 424/439; 426/73; 426/61; 426/72; 426/74; 426/383; 426/303; 426/302; 118/13

(57) ABSTRACT

An edible sticker including an edible paper, an edible ink, and an edible compound on at least a portion of the edible paper. The edible compound includes one selected from a nutrient, a supplement, a medicine, a mineral, a flavoring, a vitamin, and combinations thereof. A method of providing nutrients to a child using the edible sticker, a method of manufacturing the edible sticker, and a system for manufacturing the edible sticker have also been disclosed.

This invention relates to an edible sticker with a nutrient or other materials added. More particularly, the Invention is for a sticker, or other media, which is edible, and which can be applied to an object, wherein the sticker has been treated so as to contain or comprise added nutrients, supplements, medicines, minerals or other substances which may in some way enhance the health of the person eating the sticker.

According to one aspect of the invention, there is provided an edible sticker or other medium which has a selected added edible component, and the components may be in a combination suitable for a predetermined purpose. For example, a plurality of nutrients may be selected so as to provide energy to an athlete, or vitamins to complement a specific diet.

In one embodiment, the added component may be a nutrient. In other embodiments of the invention, the added component may be a flavoring, a medicine, a vitamin, a mineral, a nutritional supplement or the like. The invention is not limited to the type of edible component which may be added to the sticker.

Various components may be combined and grouped together on a sticker to serve a specific purpose.

The invention may be, in one aspect thereof, directed toward young adults or children. The enticement of an attractive sticker to a child may be considered as a mechanism by means of which vitamins or other nutrients may be administered to a child. In one example, an apple or other fruit may have an edible sticker of the invention applied thereto and given to a child. The child can simply eat the sticker with the apple, and at the same time receive supplements, minerals or the like while eating the apple.

### BRIEF DESCRIPTION OF THE DRAWINGS

In the drawings:

FIG. 1 is a schematic view of a sticker in accordance with one embodiment of the invention;

FIG. 2 is schematic view showing one process of applying an edible compound to a sticker; and

FIG. 3 is schematic view showing another process of applying an edible compound to a sticker, in accordance with the invention.

### DETAILED DESCRIPTION OF THE INVENTION

Reference is now made to the drawings which show various embodiments of the invention.

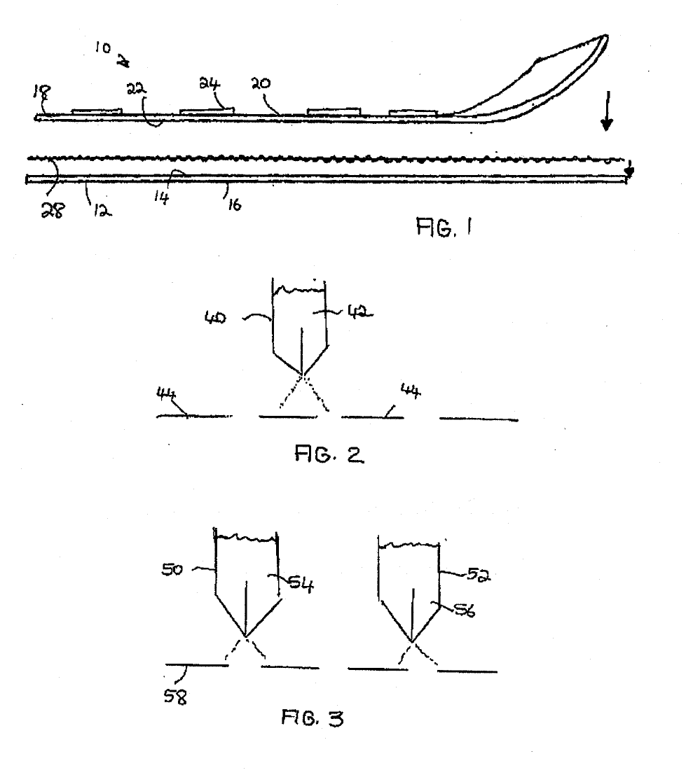

In FIG. 1 of the drawings, there is shown a schematic view of a sticker 10 constructed in accordance with one embodiment of the invention. The sticker 10 is comprised of a paper backing portion 12 having an upper surface 14 and a lower surface 16, and an edible paper portion 18 having an upper surface 20 and a lower surface 22. The upper surface 20 of the edible paper portion 18 has all or a part thereof which is covered with an edible compound 24. This edible compound may any one of a wide variety of substances comprised of but not limited to nutrients, medicines, supplements, flavorings, vitamins, minerals, and the like. A further layer may be provided on the top of the edible compound 24 to protect it from rubbing off or from becoming contaminated with foreign substances.

An edible adhesive layer 28 is applied to the lower surface 22 of the edible paper portion 22. The paper backing portion 12 covers and protects the edible adhesive layer 28 while the sticker 10 is stored, but is removed when the sticker 10 is applied to an object, such as a fruit or vegetable. When the paper backing portion 12 is removed, the edible adhesive layer 28 on the lower surface 22 of the edible paper portion 18 is exposed and is used to cause adherence of the sticker 10 to the surface of the object.

It is not necessary to remove the sticker 10 from the object before it is eaten. Since the sticker 10 is completely edible, and will typically comprise substances of use to the person eating the object, ingestion of the sticker 10 is not only not problematic, but is in fact health enhancing.

As will be appreciated, many fruits and vegetables have stickers thereon which identify their place of origin, contain bar code information for check out and pricing purposes, or display other data and facts which may be useful to the purchaser or the purveyor. In order to ensure that these stickers or labels remain on the fruit or vegetables to serve their desired purpose, they need to be reasonably securely fastened. This, however, may be a nuisance to the person eating the fruit or vegetables, since the label can often be difficult to remove and may in fact leave a residue of adhesive or other materials which may be unhealthy or at least disagreeable. This is of course off-putting to the consumer.

The advantage of the sticker of the invention is that it need not be removed at all before the fruit or vegetable is eaten. Not only is the sticker edible, but it also contains other compounds or substances which may in fact enhance the health of the person eating the fruit or vegetable. Having these compounds or substances formed directly on the sticker is a novel and effective way of administering medication, or supplementing diet with nutrients, minerals and the like. The possible combinations of substances and compounds which may be placed on the sticker is endlessly variable and specific combinations may be configured to particular needs or groups of people. The invention may be specially effective when used with children, who may be encouraged to take medicines or supplements in this unusual way. Children tend to respond to stickers and have a positive attitude toward them, making these stickers an ideal carrier for administering these substances to children

FIG. 2 of the drawings shows schematically a cartridge dispenser 40 containing a mixture solution 42 of edible ink and substance or compound including nutrients and the like. The dispenser 40 is mounted over a belt supporting a plurality of stickers 44. As each sticker 44 is moved by the belt, it receives a dispensed substance from the dispenser 40 as it passes thereunder and a layer of the substance is thus is formed on the surface thereof.

FIG. 3 of the drawings shows a situation somewhat similar to that shown in FIG. 2 of the drawings. However, the edible ink and the substance are contained in separate dispensers 50 and 52. The dispenser 50 contains edible ink 54, while dispenser 52 contains the substance 56. FIG. 3 may be useful where many types of substance are used or combined, and it is not process effective to mix the substance with the edible ink. In FIG. 3 of the drawings, the stickers 58 are sequentially moved under both the dispensers 50 and 52 and receive a dose of discharge from each dispenser as they pass beneath the dispenser.

Below are representative examples of different types of stickers with nutrient which may be made in accordance with the present invention. It should be understood that these examples are not intended to limit the scope of the invention, but rather to illustrate the diversity and variations which are possible using the edible stickers of the invention.

### EXAMPLE 1

### Printer/Vitamin Enriched

In this example, an edible sticker of the invention is created by forming a sticker as described in this specification and printing thereon using a cartridge printer an ink which comprises liquids and/or vitamins.

The following elements and components may be used to configure the edible sticker in accordance with this embodiment:

(1) Edible Ink and/or food Coloring

(2) An Ink Cartridge

(3) Liquid Vitamins and/or Minerals. These may include one of the following: Multi-Vitamin and Mineral; Essential Fatty Acids (Omega); Colostrum with Probiotics; Vitamins A, C, D, DHA; Calcium; Magnesium; Beta Carotene; and iron.

(4) Edible Base Paper.

(5) Edible Adhesive.

(6) Paper Backing.

(7) A printer which will print using the ink cartridge referenced above.

(8) Computer design program for custom stickers, for creating stickers having original or selected designs appearances.

Using the various elements as described above, the following manufacturing steps and processes may be employed to create the edible sticker of the invention:

Step 1: The Edible Ink (Food Coloring) is combined with Liquid Vitamins, to form a mixture.

Step 2: The mixture from step 1 above is added to an ink cartridge for use in a printer.

Step 3: The ink cartridge with mixture therein as described in step 2 above is loaded into or otherwise appropriately fixed in printer.

Step 4: A piece of edible paper of desired shape and size is formed, with edible adhesive backing applied on the underside of the edible paper.

Step 5: The edible paper with edible adhesive backing applied thereto is loaded into printer.

Step 6: A customized design or picture or text which may or may not be created with printing software is formed.

Step 7: The edible paper is run through the printer in a conventional fashion and during this run the cartridge ink and edible liquid nutrient mixture described above is printed on the edible paper, preferably on the upper surface thereof.

From the above, it will be appreciated that the edible Ink which may have food coloring is combined with liquid vitamins to create a vitamin enriched edible ink. This ink is loaded into a printer cartridge for use in a printer. The edible adhesive is applied to a paper backing which is then layered onto an edible base paper, in a manner similar to that used for standard labels, but comprised of edible materials. This combination is then loaded into the printer with the vitamin enriched edible ink.

Utilizing a customizable printing software program, sticker designs are created for printing. However, it will be appreciated that a software program is not necessary in this invention, although it may be preferred. The designs may be created in suitable manner such that they are able to be printed in the printer.

Vitamin enriched edible stickers are then printed and can be eaten to provide nutrients and supplements in a novel and appealing manner.

The Vitamin Enriched Edible Stickers are created in accordance with this invention and can be placed on, for example, fruits and vegetables which will increase children's consumption of these foods and improve their overall health.

### EXAMPLE 2

### Spray/Vitamin Enriched

In this example, an edible sticker of the invention is created by forming a sticker as described in this specification and spraying thereon using a spray tooling an ink which comprises liquids and/or vitamins.

The following elements and components may be used to configure the edible sticker in accordance with this embodiment:

(1) Edible Ink and/or food Coloring.

(2) An Ink Cartridge.

(3) Liquid Vitamins and/or Minerals. These may include one or more of the following: Multi-Vitamin and Mineral; Essential Fatty Acids(Omega); Colostrum with Probiotics; Vitamins A, C, D, E; DHA; Calcium; Magnesium; Beta Carotene; and Iron.

(4) Edible Base Paper.

(5) Edible Adhesive.

(6) Paper Backing.

(7) A machine which has spray tooling capabilities and which will spray liquid on a surface using the ink cartridge referenced above.

(8) Computer design program for custom stickers, for creating stickers having original or selected designs and appearances.

Using the various elements as described above, the following manufacturing steps and processes may be employed to create the edible sticker of the invention:

Step 1: The Edible Ink (Food Coloring) is combined with Liquid Vitamins, to form a mixture.

Step 2: The mixture from step 1 above is added to an ink cartridge for use in a printer.

Step 3: The ink cartridge with mixture therein as described in step 2 above is loaded into or otherwise appropriately fixed in printer.

Step 4: A piece of edible paper of desired shape and size is formed, with edible adhesive backing applied on the underside of the edible paper.

Step 5: The edible paper with edible adhesive backing applied thereto is loaded into printer.

Step 6: A customized design or picture or text which may or may not be created with printing software is formed.

Step 7: The edible paper is run through the printer in a conventional fashion and during this run the cartridge ink and edible liquid nutrient mixture described above is sprayed on the edible paper, preferably on the upper surface thereof.

Step 8: An additional spray coating may be applied onto the sticker for flavor or such other purpose selected.

From the above, it will be appreciated that the edible ink which may have food coloring is combined with liquid vitamins to create a vitamin enriched edible ink. This ink is loaded into a printer cartridge for use in a printer. The edible adhesive is applied to a paper backing which is then layered onto an edible base paper, in a manner similar to that used for standard labels, but comprised of edible materials. This combination is then loaded into the printer with the vitamin enriched edible ink.

Utilizing a customizable printing software program, sticker designs re created for printing. However, it will be appreciated that a software program is not necessary in this invention, although it may be preferred. The designs may be created in suitable manner such that they are able to be printed in the printer.

Vitamin enriched edible ink is then sprayed onto the stickers, which, may then be eaten to provide nutrients and supplements in a novel and appealing manner.

The Vitamin Enriched Edible Stickers are created in accordance with this invention and can be placed on, for example, fruits and vegetables which will increase children's consumption of these foods and improve their overall health. Furthermore, a coating may be sprayed onto Vitamins to enhance flavor and protect vitamins, for example from rubbing off or losing their effective properties.

The Vitamin Enriched Edible Stickers as described herein are created in accordance with this invention and can be placed on, for example, fruits and vegetables which will increase children's consumption of these foods and improve their overall health.

### EXAMPLE 3

### Bar Coded Stickers

In this example, an edible sticker of the invention is created by forming a sticker as described in this specification and printing thereon bar code information using a cartridge printer an ink which comprises liquids and/or vitamins.

The following elements and components may be used to configure the edible sticker in accordance with this embodiment:

(1) Edible Ink and/or food. Coloring

(2) An Ink Cartridge

(3) Liquid Vitamins and/or Minerals. These may include one or more of the following: Multi-Vitamin and Mineral; Essential Fatty Acids(Omega); Colostrum with Probiotics; Vitamins A, C, D, E; DHA; Calcium; Magnesium; Beta Carotene; and Iron.

(4) Edible Base Paper.

(5) Edible Adhesive.

(6) Paper Backing.

(7) A printer which will print using the ink cartridge referenced above.

(8) Computer design program for custom stickers, for creating stickers having original or selected designs and appearances.

(9) Bar code information.

Using the various elements as described above, the following manufacturing steps and processes may be employed to create the edible sticker of the invention:

Step 1: The Edible Ink (Food Coloring) is combined with Liquid Vitamins, to form a mixture and the bar code information.

Step 2: The mixture from step 1 above is added to an ink cartridge for use in a printer.

Step 3: The ink cartridge with mixture therein as described in step 2 above is loaded into or otherwise appropriately fixed in printer.

Step 4: A piece of edible paper of desired shape and size is formed, with edible adhesive backing applied on the underside of the edible paper.

Step 5: The edible paper with edible adhesive backing applied thereto is loaded into printer.

Step 6: A customized design or picture or text which may or may not be created with printing software is formed.

Step 7: The edible paper is run through the printer in a conventional fashion and during this run the cartridge ink and edible liquid nutrient mixture described above in step 1 is printed on the edible paper, preferably on the upper surface thereof.

From the above, it will be appreciated that the edible Ink which may have food coloring is combined with bar code information and liquid vitamins to create a vitamin enriched edible ink. This ink is loaded into a printer cartridge for use in a printer. The edible adhesive is applied to a paper backing which is then layered onto an edible base paper, in a manner similar to that used for standard labels, but comprised of edible mate. This combination is then loaded into the printer with the vitamin enriched edible ink.

Utilizing a customizable printing software program, sticker designs are created for printing. However, it will be appreciated that a software program is not necessary in this invention, although it may be preferred. The designs may be created in suitable manner such that they are able to be printed in the printer.

Vitamin enriched edible stickers are then printed and can be eaten to provide nutrients and supplements in a novel and appealing manner.

The Vitamin Enriched Edible Stickers are created in accordance with this invention and can be placed on, for example, fruits and vegetables which will increase children's consumption of these foods and improve their overall health. Furthermore, the fruits and vegetables upon which the stickers have been applied will contain bar code information, which will therefore make the consumption of these stickers safe. The fruit or other products so labeled have the dual advantage of expediting check out with the identifying bar code information, and having this information applied in a manner which allows the label to be eaten so it does not have to be removed.

### EXAMPLE 4

### Flavored Stickers

In this example, an edible sticker of the invention is created by forming a sticker as described in this specification and printing thereon using a cartridge printer an ink which comprises flavors to provide a flavored coating to the sticker.

The following elements and components ay be used to configure the edible sticker in accordance with this embodiment:

(1) Edible Ink and/or food Coloring

(2) An Ink Cartridge

(3) Flavor Coating, which may be one or more or a combination of any of the following: Caramel, Cotton Candy, Bubble Gum, Fruit Flavors, Chocolate. These are examples only, and a vast spectrum of flavors and tastes may be used.

(4) Edible Base Paper.

(5) Edible Adhesive.

(6) Paper Backing.

(7) A printer which will print using the ink cartridge referenced above.

(8) Computer design program for custom stickers, for creating stickers having original or selected designs and appearances.

Using the various elements as described above, the following manufacturing steps and processes may be employed to create the edible sticker of the invention:

Step 1: The Edible Ink (Food Coloring) is combined with flavor coating which may include, those mentioned above.

Step 2: The mixture from step 1 above is added to an ink cartridge for use in a print

Step 3: The ink cartridge with mixture therein as described in step 2 above is loaded into or otherwise appropriately fixed in printer.

Step 4: A piece of edible paper of desired shape and size is formed, with edible adhesive backing applied on the underside of the edible paper.

Step 5: The edible paper with edible adhesive backing applied thereto is loaded into printer.

Step 6: A customized design or picture or text which may or may not be created with printing software is formed.

Step 7: The edible paper is run through the printer in a conventional fashion and during this run the cartridge ink and edible flavor coating mixture described above is printed on the edible paper, preferably on the upper surface thereof.

From the above, it will be appreciated that the edible Ink which may have food coloring is combined with a flavor coating to create a vitamin enriched edible ink. This ink is loaded into a printer cartridge for use in a printer. The edible adhesive is applied to a paper backing which is then layered onto an edible base paper, in a manner similar to that used for standard labels, but comprised of edible materials. This combination is then loaded into the printer with the vitamin enriched edible ink.

Utilizing a customizable printing software program, sticker designs are created for printing. However, it will be appreciated that a software program is not necessary in this invention, although it may be preferred. The designs may be created in suitable manner such that they are able to be printed in the printer.

Edible stickers are then printed and can be eaten to provide a variety of flavorings and tastes in a novel and appealing manner.

The Vitamin Enriched Edible Stickers are created in accordance with this invention and can be placed on, for example, fruits and vegetables which will increase children's consumption of these foods to improve their overall health.

### EXAMPLE 5

### Children's Medicine

In this example, an edible sticker of the invention is created by forming a sticker as described in this specification and printing thereon using a cartridge printer an ink which comprises children's medicine (or, indeed, medicine for any person or animal).

The following elements and components may be used to configure the edible sticker in accordance with this embodiment:

(1) Edible Ink and/or food Coloring

(2) An Ink Cartridge

(3) Liquid medicine for children, adults or animals.

(4) Edible Base Paper.

(5) Edible Adhesive.

(6) Paper Backing.

(7) A printer which will print using the ink cartridge referenced above,

(8) Computer design program for custom stickers, for creating stickers having original or selected designs and appearances.

Using the various elements as described above, the following manufacturing steps and processes may be employed to create the edible sticker of the invention:

Step 1: The Edible Ink (Food Coloring) is combined with medicine, to form a mixture.

Step 2: The mixture from step 1 above is added to an ink cartridge for use in a printer.

Step 3: The ink cartridge with mixture therein as described in step 2 above is loaded into or otherwise appropriately fixed in printer.

Step 4: A piece of edible paper of desired shape and size is formed, with edible adhesive backing applied on the underside of the edible paper.

Step 5: The edible paper with edible adhesive backing applied thereto is loaded into printer.

Step 6: A customized design or picture or text which may or may not be created with printing software is formed.

Step 7: The edible paper is run through the printer in a conventional fashion and during this run the cartridge ink and edible liquid medicine mixture described above is printed on the edible paper, preferably on the upper surface thereof.

From the above, it will be appreciated that the edible ink is combined with medicine to create the medicine carrying edible ink. This ink is loaded into a printer cartridge for use in a printer. The edible adhesive is applied to a paper backing which is then layered onto an edible base paper, in a manner similar to that used for standard labels, but comprised of edible materials This combination is then loaded into the printer with the medicine added edible ink.

Utilizing a customizable printing software program, sticker designs are created for printing. However, it will, be appreciated that a software program is not necessary in this invention, although it may be preferred. The designs may be created in suitable manner such that they are able to be printed in the printer.

Edible stickers carrying the medicine are then printed and can be eaten to provide medicine requirements in a novel and appealing manner which may otherwise be more difficult to administer.

The medicated edible stickers are created in accordance with this invention and can be placed on, for example, fruits and vegetables or any other foodstuff and will make the consumption of medicines easier for kids.

### EXAMPLE 6

### Spray Vitamin Enriched

In this example, an edible sticker of the invention is created by forming a sticker as described in this specification and printing thereon using a cartridge printer an ink which comprises liquids and/or vitamins.

The following elements and components may be used to configure the edible sticker in accordance with this embodiment:

(1) Edible Ink and/or food Coloring

(2) An ink Cartridge

(3) Liquid Vitamins and/or Minerals. These may include one or more of the following: Multi-Vitamin and Mineral; Essential Fatty Acids(Omega); Colostrum with Probiotics; Vitamins A, C, D, E; DHA; Calcium; Magnesium; Beta Carotene; and Iron.

(4) Edible Base Paper.

(5) Edible Adhesive.

(6) Paper Backing.

(7) Machinery with droplet tooling.

(8) Computer design program for custom stickers, for creating stickers having original or selected designs and appearances.

Using the various elements as described above, the following manufacturing steps and processes may be employed to create the edible sticker of the invention:

Step 1: The Edible Ink (Food Coloring) is combined with Liquid Vitamins, to form a mixture.

Step 2: The mixture from step 1 above is added to an ink cartridge for use in a printer.

Step 3: The ink cartridge with mixture therein as described in step 2 above is loaded into or otherwise appropriately fixed in printer.

Step 4: A piece of edible paper of desired shape and size is formed, with edible adhesive backing applied on the underside of the edible paper.

Step 5: The edible paper with edible adhesive backing applied thereto is loaded into printer.

Step 6: A customized design or picture or text which may or may not be created with printing software is formed.

Step 7: The edible paper is run through the specialized printer and droplets of the mixture are applied on the edible paper, preferably on the upper surface thereof.

Step 8: Apply droplet coating onto sticker for flavor etc.

From the above, it will be appreciated that the edible Ink which may have food coloring is combined with liquid vitamins to create a vitamin enriched edible ink. This ink is loaded into a printer cartridge for use in a printer. The edible adhesive is applied to a paper backing which is then layered onto an edible base paper, in a mariner similar to that used for standard labels, but comprised of edible materials. This combination is then loaded into the printer with the vitamin enriched edible ink.

Utilizing a customizable printing software program, sticker designs are created for printing. However, it will be appreciated that a software program is not necessary in this invention, although it may be preferred. The designs may be created in suitable manner such that they are able to be printed in the printer.

Vitamin enriched edible ink is applied via droplets onto the stickers. A coating may be applied via droplets onto Vitamins to enhance flavor and protect vitamins.

The Vitamin Enriched Edible Stickers are created in accordance with this invention and can be placed on, for example, fruits and vegetables which will increase children's consumption of these foods and improve their overall health.

### Other Applications

(1) The stickers themselves may be comprised of multiple segments or they may have single segment. In other words, the stickers may have one face or there may be parts of the face.

(2) The matter printed on to the sticker may further have three dimensional properties.

(3) The stickers may be comprised of photo stickers.

(4) The sticker may have adhesive base backing paper. Such paper may require manual lubrication to enable them to have adhesive properties to adhere to the object, such as a fruit or vegetable.

The invention is not limited to the precise details described herein. Many variations and embodiments of the sticker may be possible and remain within the scope of the invention.

According to embodiments of the present invention, an edible sticker includes: an edible paper; an edible indicia on a first side of the edible paper; and an edible compound on at least a portion of the edible paper, the edible compound including one selected from a nutrient, a supplement, a medicine, a mineral, a flavoring, a vitamin, and combinations thereof.

According to embodiments of the present invention, a method of manufacturing an edible sticker includes: providing an edible paper; providing an edible ink; providing an edible compound, the edible compound including one selected from the group consisting of a nutrient, a supplement, a medicine, a mineral, a flavoring, a vitamin, and combinations thereof; depositing the edible ink on a first side of the edible paper; and depositing the edible compound on the first side of the edible paper.

According to embodiments of the present invention, a system for manufacturing an edible sticker includes: a first cartridge disperser for dispensing a solution of at least one of an edible ink and an edible compound; a plurality pieces of edible paper; a belt for transporting the plurality pieces of edible paper, wherein the edible compound includes one selected from the group consisting of a nutrient, a supplement, a medicine, a mineral, a flavoring, a vitamin, and combinations thereof.

According to embodiments of the present invention, a method of providing nutrients to a child including: providing an edible sticker; adapting the edible sticker to adhere to a nutritional rich item, wherein the edible sticker includes: an edible paper; an indicia on a first side of the edible paper; and an edible compound over at least a portion of the edible paper, the edible compound including one selected from the group consisting of a nutrient, a supplement, a medicine, a mineral, a flavoring, a vitamin, and combinations thereof.

In one embodiment, an edible paper includes starch. In another embodiment, an all-natural edible sticker includes sugar, hydroxypropyl methylcellulose (modified cellulose), water, natural flavor, modified corn starch, glycerin, polyglycerol esters of fatty acids, citric acid, red beet concentrate, turmeric, red cabbage extract, caramel color, and sodium bicarbonate. In another embodiment, a naturally flavored edible sticker includes tapioca starch, corn syrup solids, microcrystalline cellulose, sugar, water, canola oil, glycerine, titanium dioxide, polyglycerol esters, modified corn starch, algin, citric acid, potassium sorbate (preservative), coconut oil, soy lecithin, and locust bean gum. The naturally flavored edible sticker may further include colorings such as FD&C Blue#1 Lake, Red #40 Lake, Red #3, Yellow #5 Lake, or Yellow #6 Lake.

In one embodiment, the edible sticker includes a printed indicia. The printed indicia may include a customized design, a picture, text, a cartoon character, a bar code, or combinations thereof. The edible ink for printing the printed indicia may be deposited separately from the edible compound using a printing process. The edible ink may also be mixed with the edible compound and deposited together using a printing process, a spraying process, or combinations thereof.

In one embodiment, the edible adhesive may be an edible pressure sensitive adhesive, an edible heat activatable adhesive, or an edible water activatable adhesive. The water activatable adhesive may be activated by wetting the adhesive surface with a separate water source, such as using the drinking water, or licking using the tongue. The adhesive may also be activated as it is applied on to the nutritional rich item by the residue water on the nutritional rich item from the washing process, or from the juice of the nutritional rich item. In one embodiment, the edible paper also functions as an edible water activatable adhesive and a separate adhesive layer is not needed.

It should be understood that the example embodiments described herein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims, and equivalents thereof.

What is claimed is:

1. An edible sticker, comprising:
an edible paper;
an edible indicia on a first side of the edible paper; and
an edible compound on at least a portion of the edible paper, the edible compound comprising one selected from the group consisting of a nutrient, a supplement, a medicine, a mineral, a flavoring, a vitamin, and combinations thereof.

2. The edible sticker of claim 1, wherein the edible compound comprises at least one of liquid multi-vitamin and mineral, essential fatty acids, colostrum with probiotics, vitamin A, vitamin C, vitamin D, vitamin E, DHA, calcium, magnesium, beta carotene, iron or combinations thereof.

4. The edible sticker of claim 1, wherein the indicia comprises a customized design, a picture, text, a cartoon character, a bar code, or combinations thereof.

5. The edible sticker of claim 1, further comprising:
a protection layer over the edible compound.

6. The edible sticker of claim 1, further comprising:
a paper backing on a second side of the edible paper opposite to the first side.

7. The edible sticker of claim 6, further comprising:
an edible adhesive between the edible paper and the paper backing.

8. The edible sticker of claim 7, wherein the edible adhesive is selected from the group consisting of pressure sensitive adhesive, heat activatable adhesive, and water activatable adhesive.

9. The edible sticker of claim 8, wherein the edible adhesive is water activatable adhesive.

10. The edible sticker of claim 10, wherein the edible paper comprises the edible adhesive.

11. The edible sticker of claim 1, wherein the indicia comprises multiple colors, each of the multiple colors comprising one or more food colorings.

12. A method of providing nutrients to a child, the method comprising:
providing an edible sticker;
adapting the edible sticker to adhere to a nutritional rich item, wherein the edible sticker comprises:
an edible paper;
an indicia on a first side of the edible paper; and
an edible compound over at least a portion of the edible paper, the edible compound comprising one selected from the group consisting of a nutrient, a supplement, a medicine, a mineral, a flavoring, a vitamin, and combinations thereof.

12. The method of claim 12, wherein the nutritional rich item is a fruit or a vegetable.

13. The method of claim 12, wherein the edible sticker further comprises a water activatable adhesive on a second side of the edible paper opposite to the first side, and the method further comprising:
activating the adhesive.

14. The method of claim 13, wherein the activating the adhesive is through a liquid on the nutritional rich item and conducted currently as the act of applying.

15. A method of manufacturing an edible sticker, the method comprising:
providing an edible paper;
providing an edible ink;
providing an edible compound, the edible compound comprising one selected from the group consisting of a nutrient, a supplement, a medicine, a mineral, a flavoring, a vitamin, and combinations thereof;
depositing the edible ink on a first side of the edible paper; and
depositing the edible compound on the first side of the edible paper.

16. The method of claim 15, wherein a mixture comprising the edible ink and the edible compound are provided, and the depositing the edible ink and the depositing the edible compound are conducted in one same act.

17. The method of claim 15, wherein the depositing the edible ink and the depositing the edible compound is conducted sequentially using a printer, a spraying machine, or combinations thereof.

18. The method of claim 15, further comprising:
providing at least one of a customized design, picture, and text;
the depositing the edible ink is conducted according to the at least one of the customized design, picture, and text.

19. A system for manufacturing an edible sticker, the system comprising:
a first cartridge disperser for dispensing a solution of at least one of an edible ink and an edible compound;
a plurality pieces of edible paper;
a belt for transporting the plurality pieces of edible paper, wherein the edible compound comprises one selected from the group consisting of a nutrient, a supplement, a medicine, a mineral, a flavoring, a vitamin, and combinations thereof.

20. The system of claim 18, further comprising:
a second cartridge disperser, wherein the first cartridge is for dispensing a solution of an edible ink, and the second cartridge dispenser is for dispensing a solution of the edible compound.
